(19) United States
(12) Patent Application Publication
(10) Pub. No.: US 2026/0011450 A1
(43) Pub. Date: Jan. 8, 2026

(54) INTEGRATED PATIENT FAMILY ENGAGEMENT (PFE) AND PATIENT CENTEREDNESS INDEX ASSESSMENT

(71) Applicant: ATW Health Solutions, Olympia Fields, IL (US)

(72) Inventor: Knitasha Washington, Chicago, IL (US)

(73) Assignee: ATW Health Solutions, Olympia Fields, IL (US)

(21) Appl. No.: 19/275,343

(22) Filed: Jul. 21, 2025

Related U.S. Application Data
(63) Continuation of application No. 17/591,598, filed on Feb. 2, 2022, now abandoned.
(60) Provisional application No. 63/144,894, filed on Feb. 2, 2021.

(51) Int. Cl. G16H 50/70; G16H 10/20; G16H 15/00

(52) U.S. Cl. CPC G16H 50/70; G16H 10/20; G16H 15/00

(57) ABSTRACT

Identifying health metrics using statistical benchmark analysis are presented. Health metrics are identified using statistical models, rules and artificial intelligence, configured from a database of historical patient and hospital feedback, mapped to specific recommendations. In particular, patients can provide feedback to hospitals before, during or after a visit. As a result, assessment reports are sent to the hospital for taking action.

### CROSS REFERENCE TO RELATED APPLICATIONS

This application is a continuation of U.S. application Ser. No. 17/591,598, filed Feb. 2, 2022, which claims priority to U.S. Prov. App. No. 63/144,894, filed Feb. 2, 2021, the content of which is being hereby incorporated by reference in its entirety.

### FIELD OF THE INVENTION

The invention relates generally to computer software, and more specifically, to identifying health metrics using statistical benchmark analysis for improving patient experiences.

### SUMMARY

Methods, computer program products, and systems for identifying health metrics using statistical benchmark analysis are presented.

In one embodiment, health metrics are identified using statistical models, rules and artificial intelligence, configured from a database of historical patient and hospital feedback, mapped to specific recommendations. In particular, patients can provide feedback to hospitals before, during or after a visit. As a result, assessment reports are sent to the hospital for taking action.

Advantageously, hospitals receive custom guidance for improving patient experiences.

### BRIEF DESCRIPTION OF THE DRAWINGS

In the following drawings, like reference numbers are used to refer to like elements. Although the following figures depict various examples of the invention, the invention is not limited to the examples depicted in the figures.

FIG. 1 is a high-level block diagram illustrating a system for identifying health metrics using statistical benchmark analysis, according to one embodiment.

FIG. 2 is a high-level flow chart illustrating a method for identifying health metrics using statistical benchmark analysis, according to one embodiment.

FIG. 3 is a block diagram illustrating an example computing device, according to one embodiment.

FIGS. 4, 5 and 6 provide further embodiments of PFE evaluations and results.

### DETAILED DESCRIPTION

Methods, computer program products, and systems for identifying health metrics using statistical benchmark analysis, are described in more detail below. For the sake of conciseness, only a few embodiments are set forth, although one of ordinary skill in the art will recognize many variations within the spirit of the present invention therefrom.

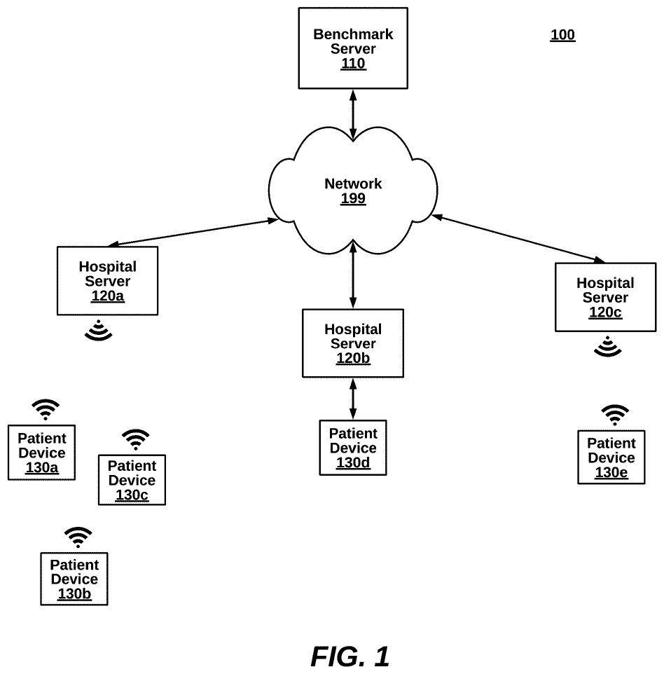

### I. Systems for Identifying Health Metrics (FIG. 1)

FIG. 1 is a high-level block diagram illustrating a system 100 for identifying health metrics using statistical benchmark analysis, according to one embodiment. The system 100 includes a benchmark server 110, hospital servers 120a-c, and patient devices 130a-e. Many other embodiments of the system 100 besides the present use case are possible. For example, the benchmark server 110 can be distributed over several physical devices, there can be a single hospital server or hundreds, and there can be a single patient device or hundreds.

The network 199 can be any data network, cellular network, wired or wireless, either currently available or developed in the future. For example, the Internet, a WLAN, a LAN, a Wi-Fi or IEEE 802.11 network, a 3G, 4G or 4G network, Bluetooth, or the like. Each of the components is directly or indirectly coupled in communication with a data communication system, or network 199, for transferring patient feedback and resulting reports.

The benchmark server 110 can be preconfigured with a database of information for generating statistical models. When a particular hospital is analyzed, a hospital profile can be generated and then compared against the statistical models. A rule set can govern what particular recommendations are made based on the comparison. In some embodiments, artificial intelligence adaptively alters the statistical models and/or the rule set itself to improve results. After the initial assessments, additional feedback can be solicited and received from hospital clients and recommendations can be updated as needed.

The hospital servers 120a-c collect responses across their patients and upload results to the benchmark server 110. Some recommendations initialize automated processes from the hospital servers. For example, an e-mail or SMS message can be automatically sent out to all of a subset of patients. In another example, hospital staff is alerted to take a specific action for a specific patient, or group of patients. In yet another example, additional information may be solicited from one or more patients based on initial responses for deeper analysis at the benchmark server 110.

The patient devices 130a-e can be terminals for patients to provide feedback to the hospital servers 120a-c. Many different configurations are possible. Patient device 130a can be a smartphone communicating over Wi-Fi with the hospital server 120a, while patient device 130d can be a personal computer of the hospital facility that is directly connected to the hospital server 120b.

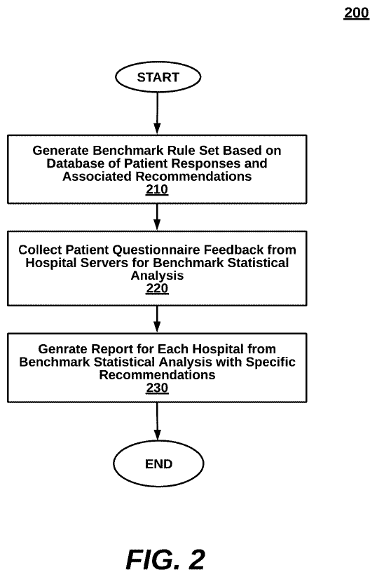

### II. Methods for Identifying Health Metrics (FIG. 2)

FIG. 2 is a high-level flow diagram illustrating a method 200 for identifying health metrics using statistical benchmark analysis, according to one embodiment. The method 200 can be implemented by, for example, the benchmark server 110 of FIG. 1. The steps of the method can be performed as shown or in different orders, with additional steps included, over various embodiments. Many of the functionalities described herein can be implemented with computer software, computer hardware, or a combination.

At step 310, a benchmark rule set is generated based on a database of patient responses and associated recommendations. The historical patient data can be actual or hypothetical.

At step 320, patient questionnaire feedback is collected form hospital servers for benchmark statistical analysis. The feedback can be prior to a visit, during a visit, or afterwards. The visit can be physical or virtual, such as through a telemedicine session over the Internet. In some cases, general information is sought from all patients, and in other cases, particular feedback is sought from targeted patients. For example, virtual visits have different circumstances from long term care visits.

At step 330, reports are generated for each hospital from the benchmark statistical analysis with specific recommendations. In some implementation, additional feedback is solicited form hospitals and/or patients, and is used for more in depth recommendations. Also, feedback can be solicited to evaluate effectiveness of the recommendations. Artificial intelligence processes can then adjust future recommendations based on effectiveness.

### III. Health Metric Details (FIG. 1)

### A. Integrated PFE Index Assessment

### Point of Care

### Original Metric Descriptions

PFE Metric 1 “Prior to admission, hospital staff provide and discuss a discharge planning checklist with every patient who has a scheduled admission, allowing for questions or comments from the patient or family (e.g., a planning checklist that is similar to CMS's Discharge Planning Checklist).”

PFE Metric 2 “Hospital conducts shift change huddles for staff and does bedside reporting with patients and family members in all feasible cases.”

### ATW Metric Description

These questions seek to understand what strategies are used at the point of care or direct care level. These are strategies that happen directly with patients during clinical encounters as well as other direct interaction with healthcare systems.
1. Inclusive discharge planning: Does your hospital have a process that provides opportunities for discussion with patients and families prior to admission where their needs, expectations and desires are discussed and included in the discharge plan?
2. Bedside Shift Change: Does your hospital do shift change reports (nurses, techs or LNA etc) at the bedside including patient and their families? Or are there other times when the care team huddles, or rounds at the bedside?
3. Are there other ways that you engage patients and families at the bedside? These would be specific strategies that are used to increase the partnership in care.

### Organizational Design, Policy, and Procedure

### Original Metric Descriptions

PFE Metric 3 “Hospital has a person or functional area, who may also operate within other roles in the hospital, that is dedicated and proactively responsible for Patient & Family Engagement and systematically evaluates PFE activities (i.e. open chart policy, PFE trainings, establishment and dissemination of PFE goals).”

PFE Metric 4 “Hospital has an active Patient and Family Engagement Committee (PFEC) OR at least one former patient that serves on a patient safety or quality improvement committee or team.”

### ATW Metric Description

These questions seek to understand what strategies are being used to engage patients and families within the organization to partner in design of processes, policies and procedures. This is the use of patient and families as advisors (PFA) within the healthcare system.
1. Strategically Implementing PFE: Does your hospital have PFE strategically placed within a department that is responsible for overseeing PFE throughout the organization?
2. Patient and Family Advisory Committee (PFAC): Does your hospital have a formal PFAC set up that meets regularly?
3. Does your hospital have PFAs on hospital committees?
4. In what other ways do PFAs play a role within the healthcare system?

### Governance

### Original Metric Description

PFE Metric 5 “Hospital has at least one or more patient(s) who serve on a Governing and/or Leadership Board and serves as a patient representative.”

### ATW Metric Description

These questions seek to understand what strategies are being used to engage patients and families at the highest level of leadership of the organization.
1. Does your hospital have patient and family representation on the governance board? This would be a full member of the board with all the same responsibilities as other members and with the inclusion of bringing the voice of patients forward.
2. What other ways do you bring the voice and perspective of patients and families to the governance level of the healthcare system.

### Community

### ATW Metric Description

These questions seek to understand what strategies are being used to engaging patients and families at the community level.
1. Does your hospital engage community members and patients in all areas of the Community Health Needs Assessment (CHNA)? This refers to having Community members and patients serve as co-leaders of the CHNA; involved in a CHNA governing council and in all planning and decision making.
2. Are there other ways that the healthcare system engages the community? This might include health fairs, community education events and other events where patients are included in the planning, implementation and evaluation.

### B. The Integrated PFE Index Assessment™

Patient-Centeredness was first identified by the Institute of Medicine as one of the six key areas of focus for improving the quality and safety of our healthcare system. Over the most recent two decades, Patient Experience has been the focal point of health care delivery experts because of its connection to reimbursement. Our research at ATW Health Solutions has identified the limitations of solely focusing on Patient Experience and examined the workstreams of Patient Family Engagement and Patient Centeredness (PFE) to understand its expanded role in quality improvement. Patient Experience, Patient Engagement and Patient Centeredness working as an integrated system is a more comprehensive framework for improving patient satisfaction, quality and reducing costs. As a result of growing research, PFE has become an increasingly accepted strategy in Quality Improvement (QI). However, to be effective there needs to be an understanding of how well PFE and QI are integrated within your organization's programs and structure and where the areas of improvement exist. The Integrated PFE Index Assessment™ makes Patient and Family Engagement (PFE) quantifiable and actionable.

The Integrated PFE Index Assessment™ evaluates how well PFE and QI are integrated within your organization and recommends areas for improvement.

The Integrated PFE Index Assessment™ was designed and validated by PFE subject-matter experts, hospital administrators and researchers who understand quality improvement. Combining the expertise of healthcare leaders, researchers, clinicians, and the lived experience of patient partners we are uniquely positioned to help our clients evolve into a more values driven organization that are patient-centered and offer the best-in-class care and quality.

The Integrated PFE Index Assessment™ includes access to an online assessment where you answer questions about your organization. Responses are analyzed using our evidence-based algorithm and validated by PFE subject matter experts to provide you a comprehensive score that addresses:
Patient and Family Advisory Council Infrastructure and Operations
PFE, Quality Improvement and Clinical Outcomes Integration
Culture of PFE and Value of the Patient's Voice

Our years of PFE research has been done in collaboration with major institutions and health systems that have validated the scientific design of The Integrated PFE Index Assessment™. Through this research we have found that having a fully integrated, effective Patient and Family Engagement program throughout an organization is associated with improvements in quality measures, such as falls and readmissions as well as overall quality and safety.

### A. Metric Description

These questions seek to understand what strategies are being used to ensure equity in care delivery.
1. Does your hospital collect REAL data (race, ethnicity, language)?
2. Does your hospital stratify clinical outcomes and diagnosis by race, ethnicity and language?
3. Does your hospital utilize the stratified data to identify populations that are having worse outcomes?
4. Does your hospital use a strategy (focus groups, individual interviews) to further identify barriers to care in the identified populations?
5. Does your hospital use the information acquired to design and improve care for all populations?
6. Are there other ways that your hospital identifies and provides care to diverse populations?

### Definition of the Integrated PFE Index Categories

1. PFE Metrics: (Propose changing this to be something like: PFE Core Measures)

These five measures provide a picture into the depth of PFE within an organization. When these PFE strategies are being used at the point of care, organizational design and governance levels it shows a commitment to PFE across the organization.
2. PFAC: This measures the level to which a PFAC is an integral part of the quality improvement strategy. Our research has found that it is not just about having a PFAC but rather how it is used, what department it reports to and how well known is it within the organization.
3. PFE Operations: This measure how and to what extent are PFA being recruited and how are their skills being utilized for maximum benefit and impact. Recruiting for diversity on all levels provides a deeper perspective and allows for higher quality improvements.
4. Leadership: Our research has shown that engaging patients and families for maximum benefit relies heavily on the support of senior leaders. This means support in terms of resources but also visibility and promotion of the PFE efforts.
5. Value of PFAs: This measure looks at how much does the organization recognize the value of PFAs? This indicates how are PFAs distributed within the organization and how much do staff rely on including PFAs on their improvement projects.
6. PFAs on Committees: This measures the extent to which PFAs are included as full members of hospital committees and work groups. It includes both the breadth and depth of the inclusion in committee work.
7. Measurement of PFE: This measures the extent that the healthcare system measures the impact that PFE is having on quality improvement and ultimately patient outcomes.

The Integrated PFE Index Assessment benchmarking report uses our scientifically tested PFE Index to measure:
Patient-Centered and Patient Engagement (PFE) Activities using 8 core areas for evaluation.
Measure how well PFE is integrated into overall health care quality

Based on the calculation of the PFE Index Score the report provides
Peer Comparisons
   Type of Hospital Facility
   Type of Geography
Recommendations
   Organization and system level “best practice” processes

### Data Collections

Survey Monkey links are extended to client for data collections.
Demographic data includes hospital name, system (if applicable), location, facility size and type, and contact information.
In addition to demographics, 25 additional questions for data collection in a variety of response types:
   Yes/No
   Check all which apply
   Free text including ‘Other-please describe’ responses
Data collection application would ideally be dynamic as research needs evolve.

### Data Analytics

Analytics occur at the individual hospital and aggregated database levels.
   165 input fields
   13 calculated fields.
   Fewer than 10 result fields→Peer ranking/quartile (or similar parameter) and other data characteristics.
Algorithms for scoring are proprietary based on ATW Health research. Each response has an empirical value associated assigned. Responses that fall outside of our assigned measures are flagged for review and validated by our team.
Qualitative summaries for action reporting are also based on scoring from research.

### Data Visualization and Reporting

Visualization of raw scores in categories as well total score.
Includes hospital results and benchmarking comparisons to peers.
   Peer data is de-identified and in aggregation
Dashboard experience that makes interpretation easy.
Scripted recommendations based on category scores.
Final data visualization for hospitals available online as well as downloadable PDF report.
Additional opportunities included trending data to see year over year comparisons.

### C. Integrated PFE Summary Report

### Integrated PFE Index Summary Report Example Hospital Academic Medical Center Urban

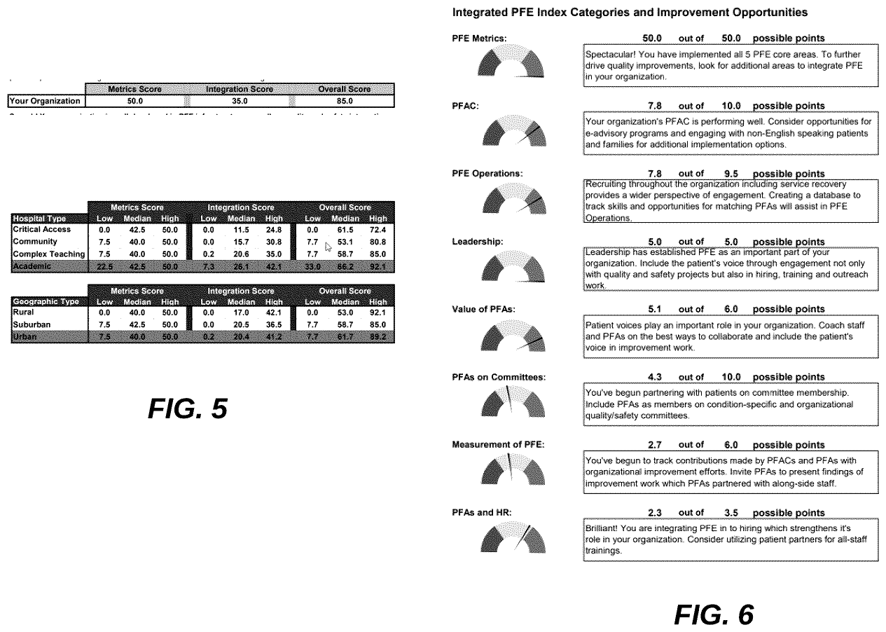

Below is a summary of your organization's scores representing your responses from the Integrated PFE Index Assessment recently completed. Organizations were scored in two areas—the Metric Score which measures implementation of patient-centered activities representing five PFE core areas. These core areas ore modeled from our years working with hospitals and align categorically with the PFE metrics developed as part of the CMS Hospital Improvement Innovation Network (HIIN) Program and represented within today's PFE literature. The Integration Score measures the depth and breadth of PFE throughout the organization and its alignment with quality and safety. The total possible score is 100 points-divided evenly between 50 possible points for the Metrics Score and 50 possible points for the Integration Score. The two scores are then added together for an Overall Score, as shown in FIG. 5.

### Peer Comparisons

To better understand your organization's performance as it relates to other participants, we are pleased to provide the following peer comparison information. The low and high scores as well as the median for the metrics, integration portions and overall score of the assessment for all the participants are provided for your reference. The peer groups for your peer group are highlighted in orange in FIG. 5.

### D. Integrated PFE Index Categories and Improvement

### Opportunities

### FIG. 6.

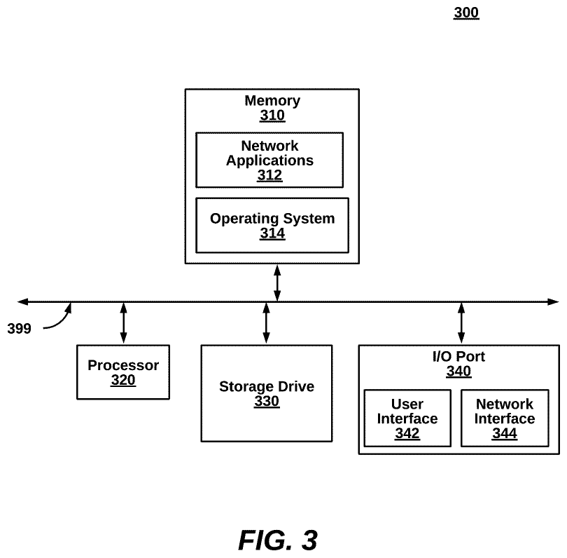

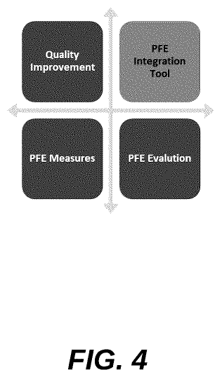

### III. Generic Computing Device (FIG. 3)

FIG. 3 is a block diagram illustrating an exemplary computing device 500 for use in the system 100 of FIG. 1, according to one embodiment. The computing device 500 is an exemplary device that is implementable for each of the components of the system 100, such as the benchmark server 110, the hospital servers 120a-c, and the patient devices 120a-e. Additionally, the computing device 300 is merely an example implementation itself, since the system 100 can also be fully or partially implemented with laptop computers, tablet computers, smart phones, Internet appliances, and the like.

The computing device 300, of the present embodiment, includes a memory 310, a processor 320, a hard drive 330, and an I/O port 340. Each of the components is coupled for electronic communication via a bus 399. Communication can be digital and/or analog, and use any suitable protocol.

Computer software products (e.g., non-transitory computer products storing source code) may be written in any of various suitable programming languages, such as C, C++, C#, Oracle® Java, JavaScript, PHP, Python, Perl, Ruby, AJAX, and Adobe® Flash®. The computer software product may be an independent application with data input and data display modules. Alternatively, the computer software products may be classes that are instantiated as distributed objects. The computer software products may also be component software such as Java Beans (from Sun Microsystems) or Enterprise Java Beans (EJB from Sun Microsystems).

Furthermore, the computer that is running the previously mentioned computer software may be connected to a network and may interface to other computers using this network. The network may be on an intranet or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system of the invention using a wireless network using a protocol such as Wi-Fi (IEEE standards 802.11, 802.11a, 802.11b, 802.11e, 802.11g, 802.11i, 802.11n, and 802.ac, just to name a few examples). For example, signals from a computer may be transferred, at least in part, wirelessly to components or other computers.

In an embodiment, with a Web browser executing on a computer workstation system, a user accesses a system on the World Wide Web (WWW) through a network such as the Internet. The Web browser is used to download web pages or other content in various formats including HTML, XML, text, PDF, and postscript, and may be used to upload information to other parts of the system. The Web browser may use uniform resource identifiers (URLs) to identify resources on the Web and hypertext transfer protocol (HTTP) in transferring files on the Web.

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1. A computer-implemented method in a benchmark server, on a data communication network, for identifying health metrics are identified using statistical models, rules and artificial intelligence, configured from a database of historical patient and hospital feedback, mapped to specific recommendations, the method comprising:
receiving feedback at the benchmark server from hospital servers before, during or after a patient visit;
generating assessment reports for patient centeredness using artificial intelligence based on the feedback analyzed against a database of actions; and
transmitting the assessment report to the hospital server for action.
